(19) United States
(12) Patent Application Publication
(10) Pub. No.: US 2026/0155241 A1
(43) Pub. Date: Jun. 4, 2026

(54) Computerized CBT Education and Training System

(71) Applicant: GAIA AG, Hamburg (DE)

(72) Inventors: Tristan Zindler, Hamburg (DE); Olaf Nackenhorst, Dortmund (DE); Bernhard Wellhöfer, Hamburg (DE); Mario Weiss, Hamburg (DE)

(21) Appl. No.: 18/966,861

(22) Filed: Dec. 3, 2024

(51) Int. Cl. G16H 40/20; G06Q 10/0631; G16H 20/70; G16H 80/00

(52) U.S. Cl. CPC G16H 40/20; G16H 20/70; G16H 80/00

(57) ABSTRACT

A system for evaluating cognitive behavioral therapy messages includes a patient messaging system; a therapist reply system; a database for storing, according to diagnosis, message and reply histories received from the patient messaging system and the therapist reply system for a plurality of patients; a computer in communication with the patient messaging system and the therapist reply system, the computer having access to the database; software executing on the computer for analyzing a message history for a particular patient to determine improvement or regression over time and/or over volume of communications through the patient messaging system and the therapist reply system; and software executing on the computer for ranking therapists by diagnosis and providing samples of higher ranked replies.

### TECHNICAL FIELD

The present disclosure relates to computers that are configured to provide interactive dialog experiences (“chat bots”), and more particularly, to improvements in the algorithms that chat bots use to provide such experiences.

### BACKGROUND

Presently, generative artificial intelligence systems (“GenAI”) are prevalent. Such systems use statistical guessing to produce a most likely correct reply to a prompt. They lack rigor in the algorithms that generate their replys, and they are prone to mistaken guesses called “hallucinations”. For specialized tasks that an LLM is not specifically trained on, there is not a high likelihood that a particular reply will be “correct” in a useful sense of that term.

An example of a specialized task is the training of Cognitive Behavioral Therapy (“CBT”) therapists. CBT sometimes may be referred to as “talk therapy.” In the CBT treatment modality, a patient or client converses with a trained professional to enhance the patient's functioning with any of a range of psychological disorders including depression, PTSD, etc.

### SUMMARY

According to aspects of the disclosure, a system for evaluating cognitive behavioral therapy messages includes a patient messaging system; a therapist reply system; a database for storing, according to diagnosis, message and reply histories received from the patient messaging system and the therapist reply system for a plurality of patients; a computer in communication with the patient messaging system and the therapist reply system, said computer having access to the database; software executing on the computer for analyzing a message history for a particular patient to determine improvement or regression over time and over volume of communications through the patient messaging system and the therapist reply system; and software executing on the computer to rank therapists by diagnosis and provide samples of higher ranked replies.

The system may be adapted to test short and long term behavior of the patient messaging system and/or the therapist reply system.

The system may be adapted to evaluate therapeutic performance of the therapist reply system.

The system may be adapted to generate synthetic training data for the patient messaging system and/or the therapist reply system.

Other features and aspects of the present teachings will become apparent from the following detailed description, taken in conjunction with the accompanying drawings, which illustrate by way of example the features in accordance with embodiments of the present teachings. The summary is not intended to limit the scope of the present teachings.

### BRIEF DESCRIPTION OF THE DRAWINGS

The present teachings are described more fully hereafter with reference to the accompanying drawings, which depict example embodiments. The following description illustrates the present teachings by way of example, not by way of limitation of the principles of the present teachings.

FIG. 1 depicts a system 100 for evaluating cognitive behavioral therapy messages, according to an aspect of the disclosure.

It should be understood that throughout the drawings corresponding reference numerals indicate like or corresponding parts and features.

### DETAILED DESCRIPTION

For purposes of explanation and not limitation, specific details are set forth such as particular structures, architectures, interfaces, techniques, etc. in order to provide a thorough understanding. In other instances, detailed descriptions of well-known devices and/or methods are omitted so as not to obscure the description with unnecessary detail.

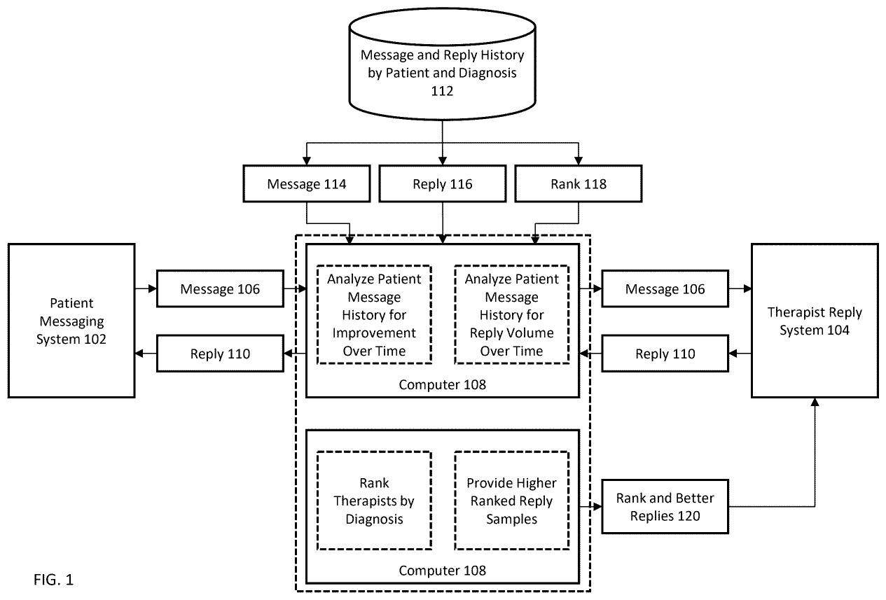

FIG. 1 depicts a system 100 for evaluating cognitive behavioral therapy messages, according to an aspect of the disclosure. The system 100 interacts between a patient (e.g., a patient messaging system) 102 and a therapist (e.g., a therapist reply system) 104. The system 100 receives a message 106 from the patient messaging system 102, processes the message 106 in a computer 108, and transmits the message 106 to the therapist reply system 104. The system 100 also receives a reply 110 from the therapist reply system 104, processes the reply 110 in the computer 108, and transmits the reply 110 to the patient messaging system 102.

The computer 108 accesses a database 112 that stores message and reply history by patient and diagnosis. From the database 112, the computer 108 retrieves messages 114, replies 116, and rankings 118. The computer 108 applies the information retrieved from the database 112 to analyze the patient message history for improvement over time and to analyze the patient message history for reply volume over time.

For example, the computer 108 may analyze the patient message history to measure relative performance of messages and therapy progress. The computer 108 may analyze the patient message history to produce an objective measure for impact of incremental changes made to the therapist reply system and/or patient messaging system. The computer 108 may analyze the patient message history to assess a patient's state, development and progress in therapy.

[Tristan, would you like to provide additional details about how the computer 108 may analyze the patient message history?]

The computer 108 may analyze the patient message history to produce various metrics, including for example composer quality (single message) or a variety of therapist metrics. Therapist metrics may be on a scale of agreement (e.g., nine bins from negative to positive) or on a numeric score between 0 and 10 or between 0 and 100, and may include answers to questions such as:
To what extent does the reply fulfill good therapeutic practice?
To what extent does the reply impact patient's emotions?
To what extent is the patient progressing in therapy? What is percentage of therapy goals completed?
Are the patient's needs met?
Are patient's resources utilized during therapy?
To what extent do the therapist and patient develop a functional relationship?

Regarding the measure “To what extent does the reply fulfill good therapeutic practice”, the computer 108 may assess each reply against key attributes of good therapeutic praxis. For example, the computer 108 may use the following eleven mostly orthogonal attributes: engaging, helpful, language, empathic, actionable, relevant, accurate, appropriate, accepting, clear, empowering. These may be assessed based on natural language processing (NLP) of each reply, e.g., by latent semantic analysis (LSA), to categorize each reply into one of a plurality of grades (e.g., nine grades) of agreement/disagreement with each of the attributes.

Regarding the measure “To what extent does the reply impact patient's emotions?”, the computer 108 may employ natural language processing, such as LSA, to assess the patient's emotional spectrum, development, and activation. Semantic space theory (SST) may be adapted to measure a full spectrum of emotions, e.g., 52 different emotions. The 52 emotions are: [‘admiration’, ‘adoration’, ‘aesthetic_appreciation’, ‘amusement’, ‘anger’, ‘annoyance’, ‘anxiety’, ‘awe’, ‘awkwardness’, ‘boredom’, ‘calmness’, ‘concentration’, ‘confusion’, ‘contemplation’, ‘contempt’, ‘contentment’, ‘craving’, ‘desire’, ‘determination’, ‘disappointment’, ‘disapproval’, ‘disgust’, ‘distress’, ‘doubt’, ‘ecstasy’, ‘embarrassment’, ‘empathic_pain’, ‘enthusiasm’, ‘entrancement’, ‘envy’, ‘excitement’, ‘fear’, ‘gratitude’, ‘guilt’, ‘horror’, ‘interest’, ‘joy’, ‘love’, ‘nostalgia’, ‘pain’, ‘pride’, ‘realization’, ‘relief’, ‘romance’, ‘sadness’, ‘sarcasm’, ‘satisfaction’, ‘shame’, ‘surprise_negative’, ‘surprise_positive’, ‘sympathy’, ‘tiredness’, ‘triumph’] which is based on the Semantic Space Theory as published in https://www.cell.com/trends/cognitive-sciences/abstract/S1364-6613(20)30276-X.

SST measurements may be purely text-driven on a scale [0,10] with chain-of-thoughts. Emotions measured by SST may be compared to expert reference, emotional activation and shifts. The concept of shifts is that by tracking the strength of emotions over time certain emotions might be activated during therapy or the person's emotional spectrum might change significantly over time (emotion shift), e.g. from sadness to acceptance during grieving. Both are indicators for therapeutic progress.

Regarding the measures “To what extent is the patient progressing in therapy? What is percentage of therapy goals completed?” The computer 108 may implement a complex LLM goal generator to define N short/mid/long-term SMART (Specific, Measurable, Achievable, Relevant, and Time-Bound) goals obtained by NLP of the therapist replies and patient messages. For a progress indicator on each goal, the computer 108 may estimate fulfilled percentage of each goal. Progress indicators are goal specific and are defined when defining the specific goals for the patient. For example, if the goal is to reduce social anxiety, progress indicators might be increased social interactions and reduced avoidance of social situations as well as lower self-reported anxiety levels in social settings. Based on averaged progress toward therapy goals, the computer 108 may estimate total therapy completion. Although this would be an estimate, not an exact figure, clear trends may be visible.

Regarding the question, “Are the patient's needs met?” The computer 108 may analyze the patient-therapist interaction using, for example, an encoder network, a transformer network, latent semantic analysis, semantic space theory, or other natural language processing or large language model techniques. One purpose of analyzing the interaction is to generate answers to questions about physiological needs, safety needs, feelings of love and belonging, feelings of esteem, actions toward self-actualization, expressions of autonomy, expressions of relatedness, and actions demonstrating emotional competence. The computer then may categorize answers into 9 grades of dis/agreement to produce a score for each question between [−1, 1]. Another purpose of analyzing the interaction is to identify missing information, e.g., clinically appropriate discussions that have not occurred between the patient and therapist.

Regarding the question, “Are patient's resources utilized during therapy?” The computer 108 may analyze the patient-therapist interaction using, for example, an encoder network, a transformer network, latent semantic analysis, semantic space theory, or other natural language processing or large language model techniques. One purpose of analyzing the interaction is to identify incidents of communication that indicate engagement of at least the following resource categories: completeness, diversity, sufficiency for therapy, activation, frequency, integration into therapy strategy, impact on patients progress, and activation of identified resources. Categories of resources include essential resources such as emotional, cognitive, social, physical, financial, spiritual, environmental, and educational/vocational resources. The completeness, diversity, sufficiency for therapy, activation, activation frequency of these resources are evaluated as well as the therapist role in resource reinforcement, the integration of resources into therapy strategy and the impact of patient's engagement and progress of the resource. The analysis is performed by an LLM based on the full history of the therapy conversation.

Regarding the question, “To what extent do the therapist and patient develop a functional relationship?” The computer 108 may analyze the patient-therapist interaction using, for example, an encoder network, a transformer network, latent semantic analysis, semantic space theory, or other natural language processing or large language model techniques. One purpose of analyzing the interaction is to identify incidents of communication that indicate performance by the therapist and/or patient. The therapeutic relationship is evaluated in categories such as effectiveness, goal alignment, emotional attunement, communication clarity, cultural sensitivity, personalization, progress reflection, supportiveness, crisis management, therapeutic alliance, empathy, validation, approach alignment and flexibility. Each category is briefly analyzed by an LLM summarizing findings, key supporting evidence, notable patterns or trends, and actionable insights. A specific question for each category is evaluated on a nine-graded scale of agreement and converted into a numerical performance metric.

Thus, the system 100 can implement a “learning loop” in which a therapist 104 (optionally, a digital therapist as described in commonly owned patent application with attorney docket 07656-P0037A) interacts with a patient 102 (optionally, a digital patient as described in commonly owned patent application with attorney docket 07656-P0038A), and the system 100 intermediates or monitors the interaction and evaluates the interaction using developed metrics. The interaction between therapist and patient generates valuable training data, principally for use by other therapists but also useful for the therapist 104. Where the therapist and/or patient are digital, the system 100 provides for a self-learning mechanism with continuous feedback loop. Thus, the system 100 captures and logs interactions between therapist and patient, evaluates the captured data using the developed metrics, and then fine-tunes the digital therapist and/or patient based on the evaluation. The computer continues to evaluate the fine-tuned model, creating a continuous improvement loop, which contributes to the development of increasingly sophisticated digital models for therapy.

The system 100 also may rank therapists by diagnosis, that is compiling ratings of therapists'effectiveness categorized by diagnoses of their patients. The system 100 also may provide higher ranked or higher rated therapist responses 120 as feedback to the therapist or therapist reply system 104.

The present teachings have been described in language more or less specific as to structural, mechanical, and functional features. It is to be understood, however, that the present teachings are not limited to the specific features shown and described, since the apparatus, system, and/or method herein disclosed comprises preferred forms of putting the present teachings into effect.

Generally, all terms used in the claims are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. The use of “first”, “second,” etc. for different features/components of the present disclosure are only intended to distinguish the features/components from other similar features/components and not to impart any order or hierarchy to the features/components, unless explicitly stated otherwise. The phrase “at least one of,” when used with a list of items, means that different combinations of one or more of the listed items may be used, and only one item in the list may be needed. For example, “at least one of: A, B, and C” includes any of the following combinations: A; B; C; A and B; A and C; B and C; and A and B and C.

Other than in the operating examples, or where otherwise indicated, all numbers expressing quantities of ingredients or reaction conditions used herein are to be understood as modified in all instances by the term “about”.

While the present teachings have been described above in terms of specific embodiments, it is to be understood that they are not limited to those disclosed embodiments. Many modifications and other embodiments will come to mind to those skilled in the art to which this pertains, and which are intended to be and are covered by both this disclosure and the appended claims. For example, in some instances, one or more features disclosed in connection with one embodiment can be used alone or in combination with one or more features of one or more other embodiments. It is intended that the scope of the present teachings should be determined by proper interpretation and construction of any claims and their legal equivalents, as understood by those of skill in the art relying upon the disclosure in this specification and the attached drawings.

What is claimed is:

1. A system for evaluating cognitive behavioral therapy messages, comprising:
a patient messaging system;
a therapist reply system;
a database for storing, according to diagnosis, message and reply histories received from the patient messaging system and the therapist reply system for a plurality of patients;
a computer in communication with the patient messaging system and the therapist reply system, said computer having access to the database;
software executing on the computer for analyzing a message history for a particular patient to determine improvement or regression over time and/or over volume of communications through the patient messaging system and the therapist reply system; and
software executing on the computer for ranking therapists by diagnosis and providing samples of higher ranked replies.

2. The system of claim 1, wherein the software for ranking therapists implements a semantic analysis technique on a plurality of sequences of messages and replies, each of the sequences being associated with a patient and a therapist.

3. The system of claim 2, wherein the semantic analysis technique is latent semantic analysis (LSA).

4. The system of claim 2, wherein the semantic analysis technique uses semantic space theory (SST).

5. The system of claim 2, wherein the software for ranking therapists analyzes the message history to assess a patient's state, development, and/or progress in therapy.

6. The system of claim 5, wherein the software ranks a plurality of therapists based on respective assessments of state, development, and/or progress in therapy for one or more patients associated with each of the plurality of therapists.

7. The system of claim 1, wherein the software for ranking therapists analyzes the message history using a semantic technique to assess each reply against key attributes of therapeutic praxis, said attributes including at least two of: engaging, helpful, language, empathic, actionable, relevant, accurate, appropriate, accepting, clear, empowering.

8. The system of claim 1, wherein the software for ranking therapists analyzes the message history using a semantic technique to assess at least one of the patient's emotional spectrum, development, and activation.

9. The system of claim 1, wherein the software for ranking therapists provides higher ranked or higher rated therapist responses as feedback to the therapist reply system.

10. The system of claim 1, wherein the software executing on the computer is adapted to generate synthetic training data for the patient messaging system and/or the therapist reply system.
